(19) United States
(12) Patent Application Publication
(10) Pub. No.: US 2017/0348261 A1
(43) Pub. Date: Dec. 7, 2017

(54) MEDICAL DERMATOLOGICAL PREPARATION FOR EXTERNAL USE

(71) Applicant: NIPRO CORPORATION, Osaka-shi, Osaka (JP)

(72) Inventors: Noriko NAKAJIMA, Osaka-shi (JP); Takehumi OFUSA, Osaka-shi (JP)

(73) Assignee: NIPRO CORPORATION, Osaka-shi, Osaka (JP)

(21) Appl. No.: 15/539,477

(22) PCT Filed: Dec. 24, 2015
(86) PCT No.: PCT/JP2015/086051

§ 371 Date: Jun. 23, 2017

(30) Foreign Application Priority Data
Dec. 26, 2014 (JP) 2014-266527

(51) Int. Cl. A61K 31/192; A61K 9/06; A61K 47/44; A61K 47/28; A61K 47/14; A61K 47/10; A61K 47/06; A61K 9/107; A61K 8/06; A61K 9/00; A61K 8/92; A61K 8/86; A61K 8/63; A61K 8/37; A61K 8/368; A61K 8/34; A61K 8/31; A61Q 19/00

(52) U.S. Cl. CPC A61K 31/192; A61Q 19/007; A61K 9/06; A61K 47/44; A61K 47/28; A61K 47/14; A61K 47/10; A61K 47/06; A61K 9/107; A61K 8/06; A61K 9/0014; A61K 8/92; A61K 8/86; A61K 8/63; A61K 8/375; A61K 8/368; A61K 8/342; A61K 8/31; A61K 2800/75

(57) ABSTRACT

The present invention relates to a medical dermatological preparation for external use comprising an active drug, polyoxyethylene arachyl ether, stearyl alcohol, a liquid oily ingredient, a moisturizing ingredient and water, and provides a medical dermatological preparation for external use, which reduces occurrence or degree of a side effect such as skin irritation in the medical dermatological preparation for external use. The present invention provides particularly a medical dermatological preparation for external use, which comprises adapalene and reduces occurrence and degree of a side effect such as skin irritation.

### TECHNICAL FIELD

The present invention relates to a medical dermatological preparation for external use, in particular to a medical dermatological preparation for external use which can reduce skin irritation by a moisturizing effect thereof.

### BACKGROUND ART

Generally it is known that a medical dermatological preparation for external use causes skin irritation such as skin dryness and skin discomfort (a tingling in skin, or the like) as a side effect. For example, in the case of adapalene gel which is placed on the market as a trade name: Differin Gel 0.1%, it has been reported that skin irritation being after application to the skin occurs frequently for a time period of two weeks after starting the use thereof (Non-patent Document 1). Further, the same skin irritation has been reported also in the case of a tacrolimus ointment.

Examples of symptoms of skin irritation when the adapalene gel is used include the skin discomfort (the tingling in skin, or the like), skin dryness, erythema, desquamation and itching, and it has been reported that these symptoms of skin irritation are alleviated by a combination use with a moisturizing agent commercially available as the trade name: Cetaphil (registered trade mark) lotion (Non-patent Document 2). It is noted that the Cetaphil (registered trade mark) lotion is a moisturizing lotion comprising water, glycerin, hydrogenated polyisobutene, Ceteareth-20, cetearyl alcohol, macadamia nut oil, tocopherol acetate, dimethicone, benzyl alcohol, phenoxyethanol, panthenol, stearoxytrimethylsilane, farnesol, stearyl alcohol, (acrylates/alkyl acrylate (C10-30)) crosspolymer, sodium hydroxide, citric acid and the like.

### PRIOR ART DOCUMENT

### Non-Patent Document

Non-Patent Document 1: Medical & Drug Journal, 2010, Vol. 46, No. 2, pp. 647-649
Non-Patent Document 2: Journal of Dermatological Treatment, 2013; 24: 278-282

### SUMMARY OF THE INVENTION

### Problem to be Solved by the Invention

However, in the case of a combination use with a moisturizing agent, there is still room for improvement with respect to an alleviating effect thereof on symptoms of skin irritation. Further, there is a case where a difference in the alleviating effect arises or a sufficient effect cannot be obtained depending on how to combine a moisturizing agent, and furthermore, there is a problem that a patient's compliance for the combination use cannot be fully obtained since the use of two formulations is troublesome.

Thus, an object of the present invention is to provide a medical dermatological preparation for external use which reduces occurrence or degree of a side effect such as skin irritation in the medical dermatological preparation for external use.

### Means to Solve the Problem

The inventors of the present invention have made intensive studies in the light of the above-mentioned problems, and as a result, have found that the occurrence and degree of the skin irritation can be reduced by compounding polyoxyethylene arachyl ether, stearyl alcohol, a liquid oily ingredient, a moisturizing ingredient and water in addition to an active drug without a combination use of other moisturizing preparation, and have completed the present invention.

Namely, the present invention relates to:

[1] a medical dermatological preparation for external use comprising an active drug, polyoxyethylene arachyl ether, stearyl alcohol, a liquid oily ingredient, a moisturizing ingredient and water,
[2] the medical dermatological preparation for external use of the above [1], wherein the active drug is adapalene,
[3] the medical dermatological preparation for external use of the above [1] or [2], wherein the liquid oily ingredient is at least one selected from the group consisting of squalane, liquid paraffin, jojoba oil, isopropyl myristate and isopropyl palmitate,
[4] the medical dermatological preparation for external use of any of the above [1] to [3], wherein the moisturizing ingredient is at least one selected from the group consisting of glycerin, 1,3-butylene glycol, dipropylene glycol, polyethylene glycol, sorbitol, urea, glycolic acid, heparinoids, pyrrolidone carboxylic acid, collagen, γ-orizanol, γ-linolenic acid, linoleic acid, vitamin E, vitamin D, vitamin A, cholesterol, glucosamine, sodium hyaluronate, sodium chondroitin lactate, casein, glucose, fructose, trehalose, maltose, pullulan, erythritol, hydrolyzed fibroin, hydrolyzed collagen, maltitol and saccharose,
[5] the medical dermatological preparation for external use of any of the above [1] to [4], further comprising an emulsion stabilizer,
[6] the medical dermatological preparation for external use of the above [5], wherein the emulsion stabilizer is at least one selected from the group consisting of cetanol, cetostearyl alcohol, behenyl alcohol, batyl alcohol, batyl isostearate and batyl monostearate, and
[7] the medical dermatological preparation for external use of any of the above [1] to [6], which is in a dosage form of a cream, a gel or a lotion.

### Effects of the Invention

The present invention can provide a medical dermatological preparation for external use which can reduce efficiently occurrence and degree of skin irritation being attributable to an active drug by using single formulation prepared by combining the active drug with polyoxyethylene arachyl ether, stearyl alcohol, a liquid oil ingredient, a moisturizing ingredient and water.

### BRIEF DESCRIPTION OF THE DRAWINGS

FIG. 1 is a graph showing a high-frequency conductance of a skin surface corneum.

FIG. 2 is a graph showing a high-frequency conductance of a skin surface corneum.

FIG. 3 is a graph showing a high-frequency conductance of a skin surface corneum.

FIG. 4 is a photographic image of a medical dermatological preparation for external use of one embodiment of the present invention taken by a polarizing microscope.

FIG. 5 is a photographic image of a conventional medical dermatological preparation for external use taken by a polarizing microscope.

### EMBODIMENT FOR CARRYING OUT THE INVENTION

The present invention relates to a medical dermatological preparation for external use comprising an active drug, polyoxyethylene arachyl ether, stearyl alcohol, a liquid oily ingredient, a moisturizing ingredient and water as essential ingredients.

In the present invention, the active drug is not limited particularly as long as it is a compound desired to alleviate skin irritation, and examples thereof include steroid, a therapeutic agent for psoriasis and atopy (tacrolimus), an antibiotic, adapalene, a compound drug of adapalene and benzoyl peroxide, phenothrin, bimatoprost and the like. Particularly preferred are adapalene and a compound drug of adapalene and benzoyl peroxide.

A content of the active drug can be set easily by a person skilled in the art according to kind and physical property of the active drug to be used, dosage form and kinds and amounts of a dissolving agent, a stabilizing agent and an anti-oxidizing agent. For example, in the case where adapalene is used as the active drug and a cream is selected as a dosage form, usually an amount of adapalene is preferably from 0.01 to 1.0% by mass, more preferably from 0.1 to 0.3% by mass based on the total amount of the medical dermatological preparation for external use.

A content of polyoxyethylene arachyl ether is not limited particularly, and is preferably from 0.1 to 20% by mass, more preferably from 0.2 to 10% by mass based on the total amount of the medical dermatological preparation for external use. Polyoxyethylene arachyl ether is a nonionic surfactant. When polyoxyethylene arachyl ether is compounded in an amount of more than 20% by mass, there is a tendency that a trouble with skin irritation arises. Further, when the content of polyoxyethylene arachyl ether is less than 0.1% by mass, there is a tendency that emulsification easily becomes unstable, separation of an aqueous phase and an oil phase occurs and a problem with a quality arises.

A content of stearyl alcohol is not limited particularly, and is preferably from 0.1 to 20% by mass, more preferably from 0.2 to 10% by mass based on the total amount of the medical dermatological preparation for external use. When stearyl alcohol is compounded in an amount of more than 20% by mass, there is a tendency that a problem with a quality arises, such as a too high viscosity, decrease in smoothness of a surface of the preparation and insufficient spreading of the preparation on a skin surface. Further, when the content of stearyl alcohol is less than 0.1% by mass, a problem with a quality arises due to a too low viscosity.

Further, in the present invention, polyoxyethylene arachyl ether and stearyl alcohol can be used as a mixture thereof mixed in various ratios. A content of the mixture of polyoxyethylene arachyl ether and stearyl alcohol is not limited particularly, and is preferably from 0.1 to 20% by mass, more preferably from 1 to 15% by mass, further preferably from 5 to 10% by mass based on the total amount of the medical dermatological preparation for external use. When the mixture of polyoxyethylene arachyl ether and stearyl alcohol is compounded in an amount of more than 20% by mass, there is a tendency that a problem with skin irritation arises, and a problem with a quality such as a too high viscosity arises, thereby decreasing smoothness of a surface of the preparation and causing insufficient spreading of the preparation on a skin surface. Further, when the content of the mixture of polyoxyethylene arachyl ether and stearyl alcohol is less than 0.1% by mass, an amount of lamellar liquid crystals formed in the preparation is too small and there is a tendency that a moisturizing effect is hardly exhibited.

In the present invention, examples of the usable liquid oily ingredient include hydrocarbon oil, animal and vegetable oils, fatty acid ester oil, branched unsaturated higher fatty acid, branched unsaturated higher alcohol, silicon oil and the like. Specifically examples of hydrocarbon oil include squalane, liquid paraffin and the like; examples of animal and vegetable oils include jojoba oil, macadamia nut oil, rose hip oil, corn oil, olive oil, castor oil, almond oil, sunflower oil, sesame oil, safflower oil, grape seed oil, soybean oil and the like; examples of fatty acid ester oil include isopropyl myristate, octyldodecyl myristate, isopropyl palmitate, diethyl sebacate, diisopropyl sebacate, diisopropyl adipate, ethyl oleate, hexadecyl isostearate, cetyl 2-ethyl hexanoate, propylene glycol caprylate, propylene glycol dicaprylate, glyceryl tri(2-ethylhexanoate), decanoyl/octanoyl-glycerides and the like; examples of branched unsaturated higher fatty acid include isostearic acid, oleic acid, linoleic acid and the like; examples of branched unsaturated higher alcohol include octyldodecanol, 2-hexyl decanol, oleyl alcohol and the like, and from the viewpoint of permeability into a skin, oxidative stability and easy spreading on a skin, squalane is used preferably. These liquid oily ingredients may be used alone, and can be used in combination of two or more thereof.

A content of the liquid oil ingredient is not limited particularly, and is preferably from 3 to 30% by mass, more preferably from 5 to 10% by mass based on the total amount of the medical dermatological preparation for external use.

In the present invention, examples of the usable moisturizing ingredient include glycerin, 1,3-butylene glycol, dipropylene glycol, polyethylene glycol, sorbitol, urea, glycolic acid, heparinoids, pyrrolidone carboxylic acid, collagen, γ-orizanol, γ-linolenic acid, linoleic acid, vitamin E, vitamin D, vitamin A, cholesterol, glucosamine, sodium hyaluronate, sodium chondroitin lactate, casein, glucose, fructose, trehalose, maltose, pullulan, erythritol, hydrolyzed fibroin, hydrolyzed collagen, maltitol and saccharose, and from the viewpoint of high moisture retention, long-lasting moisture retention and economy, glycerin is used preferably. These moisturizing ingredients may be used alone, and can be used in combination of two or more thereof.

A content of the moisturizing ingredient is not limited particularly, and is preferably from 1 to 60% by mass, more preferably from 3 to 40% by mass based on the total amount of the medical dermatological preparation for external use.

Further, it is preferable that the medical dermatological preparation for external use of the present invention contains an emulsion stabilizer in order to inhibit collapsing of emulsified particles and hold the emulsified particles on a skin for a long period of time, thereby enhancing moisture retention and making effects of inhibiting occurrence of and reducing a side effect more efficiently. The emulsion stabilizer is also known as an emulsification stabilizer in this technical field and is not limited particularly. Examples thereof include cetanol, cetostearyl alcohol, behenyl alcohol, batyl alcohol, batyl isostearate, batyl monostearate and the like, and batyl isostearate and batyl monostearate are used preferably. These emulsion stabilizers may be used alone, and can be used in combination of two or more thereof.

When the emulsion stabilizer is used, a content thereof is not limited particularly, and is preferably from 0.1 to 20% by mass, more preferably from 0.5 to 10% by mass based on the total amount of the medical dermatological preparation for external use.

In addition to the above ingredients, in the medical dermatological preparation for external use of the present invention, it is possible to compound various additives usually used in this technical field as required within a range not to impair the effects of the present invention, such as other surfactants, a stabilizing agent, a thickener, an antiseptic agent, an anti-oxidizing agent, a pH regulator, a dye, a pigment and a perfume.

Examples of the other surfactants include polyoxyethylene hydrogenated castor oil, sorbitan monostearate, sorbitan monopalmitate, glycerin monostearate, sorbitan monolaurate, a polyoxyethylene-polyoxypropylene block copolymer, polysorbates, sodium lauryl sulfate, sucrose fatty acid ester, lecithin and the like. Examples of the stabilizing agent include edetates and the like. Examples of the thickener include a carboxyvinyl polymer, hydroxypropyl methylcellulose, hydroxyethyl cellulose, hydroxypropyl cellulose and the like. Examples of the antiseptic agent include alkylparabens such as methylparaben and propylparaben, phenoxyethanol, thymol and the like. Examples of the anti-oxidizing agent include sodium hydrogen sulfite, ascorbic acid, tocopherol, dibutylhydroxytoluene benzotriazole and the like. Examples of the pH regulator include organic acids and inorganic acids such as citric acid, acetic acid, tartaric acid, malic acid, lactic acid, hydrochloric acid and phosphoric acid; an inorganic base such as sodium hydroxide; organic amines such as diisopropanolamine and triethanolamine; and the like. These additives may be used alone, and can be used in combination of two or more thereof.

The amounts of the additives such as surfactants, a stabilizing agent, a thickener, an antiseptic agent, an anti-oxidizing agent and a pH regulator can be set adequately by a person skilled in the art according to kinds of additives to be used.

The dosage form of the medical dermatological preparation for external use of the present invention is not limited particularly, and examples thereof include embrocations such as external solid agents (a powder for external use), external liquid agents (a liniment, a lotion), spraying agents (an aerosol, a pump spray for external use), a cream and a gel. A cream, a gel and a lotion are preferable, and a cream is more preferable from the viewpoint that a sustained moisturizing effect can be expressed and the active drug can be maintained stably in a uniformly suspended and dispersed state for a long period of time. These preparations can be prepared by a usual method.

For preparing the cream, for example, purified water is added to an active drug, a moisturizing ingredient, and according to necessity, a thickener, a water-soluble antiseptic agent, a stabilizing agent and an anti-oxidizing agent and the like, and followed by heating to make an aqueous phase. Next, as oily ingredients polyoxyethylene arachyl ether, stearyl alcohol, a liquid oily ingredient, and according to necessity, an emulsion stabilizer, a fat-soluble antiseptic agent and anti-oxidizing agent are heated and dissolved to make an oil phase. The obtained aqueous phase and the obtained oil phase are subjected to mixing by stirring (emulsification) with heating, and then, as needed, a pH regulator and the like are added, followed by stirring with cooling. Thus, the cream can be prepared.

Other dosage forms such as external solid agents (a powder for external use), external liquid agents (a liniment, a lotion), spraying agents (an aerosol, a pump spray for external use), and a gel can also be prepared by a known method prevailing in a field of each of preparations of the respective dosage forms.

The present invention is then explained below in detail by means of Examples and Comparative Examples, but is not limited to these Examples.

### EXAMPLE

Ingredients used in Examples and Comparative Examples are those described in Japanese Pharmacopoeia or Japanese Pharmaceutical Excipients.

### Example 1

0.1% by mass of adapalene, 0.2% by mass of methylparaben, 20% by mass of glycerin, 0.3% by mass of a carboxyvinyl polymer, 0.1% by mass of disodium edetate hydrate and a proper amount of purified water were mixed and heated at a temperature of 80° C. or higher (aqueous phase). Next, 5% by mass of a mixture of polyoxyethylene arachyl ether and stearyl alcohol (WAX230 available from Nikko Chemicals Co., Ltd.), 5% by mass of squalane and 0.1% by mass of propylparaben were mixed and dissolved by heating to a temperature of 80° C. or higher (oil phase). The oil phase was added to the aqueous phase in the state that is heated at 80-90° C. and stirred with a homogenizing mixer (manufactured by PRIMIX Corporation), followed by stirring for three minutes at 3500 rpm to emulsify. Then, a solution obtained by adding and dissolving 0.045% by mass of sodium hydroxide in a proper amount of purified water was added to the mixture under stirring using a paddle mixer (manufactured by Nikko Chemicals Co., Ltd.). A purified water was added up to 100% by mass of the total amount of the preparation, and the mixture was stirred at room temperature until cool to 30° C. or lower.

The obtained preparation was in a form of a cream and it was confirmed with a polarizing microscope that the preparation had a lamellar liquid crystal structure.

### Example 2

0.1% by mass of adapalene, 0.2% by mass of methylparaben, 30% by mass of glycerin, 0.3% by mass of a carboxyvinyl polymer, 0.1% by mass of disodium edetate hydrate and a proper amount of purified water were mixed and heated at a temperature of 80° C. or higher (aqueous phase). Next, 5% by mass of a mixture of polyoxyethylene arachyl ether and stearyl alcohol (WAX230 available from Nikko Chemicals Co., Ltd.), 0.1% by mass of propylparaben and 10% by mass of squalane were mixed and dissolved by heating at a temperature of 80° C. or higher (oil phase). The oil phase was added to in the state that is heated at 80-90° C. and stirred with a homogenizing mixer (manufactured by PRIMIX Corporation), followed by stirring for three minutes at 3500 rpm to emulsify. Then, a solution obtained by adding and dissolving 0.045% by mass of sodium hydroxide in a proper amount of purified water was added to the mixture under stirring using a paddle mixer (manufactured by Nikko Chemicals Co., Ltd.). A purified water was added up to 100% by mass of the total amount of the preparation, and the mixture was stirred at room temperature until cool to 30° C. or lower.

The obtained preparation was in a form of a cream and it was confirmed with a polarizing microscope that the preparation had a lamellar liquid crystal structure.

### Example 3

0.1% by mass of adapalene, 0.1% by mass of methylparaben, 25% by mass of glycerin, 0.3% by mass of a carboxyvinyl polymer, 0.03% by mass of disodium edetate hydrate and a proper amount of purified water were mixed and heated at a temperature of 80° C. or higher (aqueous phase). Next, 8% by mass of a mixture of polyoxyethylene arachyl ether and stearyl alcohol (WAX230 available from Nikko Chemicals Co., Ltd.), 0.1% by mass of propylparaben and 5% by mass of squalane were mixed and dissolved by heating at a temperature of 80° C. or higher (oil phase). The oil phase was added to aqueous phase in the state that is heated at 80-90° C. and stirred with a homogenizing mixer (manufactured by PRIMIX Corporation), followed by stirring for three minutes at 3500 rpm to emulsify. Then, a solution obtained by adding and dissolving 0.03% by mass of sodium hydroxide in a proper amount of purified water was added to the mixture under stirring using a paddle mixer (manufactured by Nikko Chemicals Co., Ltd.). A purified water was added up to 100% by mass of the total amount of the preparation, and the mixture was stirred at room temperature until cool to 30° C. or lower.

The obtained preparation was in a form of a cream and it was confirmed with a polarizing microscope that the preparation had a lamellar liquid crystal structure.

### Example 4

0.1% by mass of adapalene, 0.1% by mass of methylparaben, 25% by mass of glycerin, 0.4% by mass of a carboxyvinyl polymer, 0.03% by mass of disodium edetate hydrate and a proper amount of purified water were mixed and heated at a temperature of 80° C. or higher (aqueous phase). Next, 5% by mass of a mixture of polyoxyethylene arachyl ether and stearyl alcohol (WAX230 available from Nikko Chemicals Co., Ltd.), 0.1% by mass of propylparaben, 0.1% by mass of cholesterol and 5% by mass of squalane were mixed and dissolved by heating at a temperature of 80° C. or higher (oil phase). The oil phase was added to the aqueous phase in the state that is heated at 80-90° C. and stirred with a homogenizing mixer (manufactured by PRIMIX Corporation), followed by stirring for three minutes at 3500 rpm. Then, a solution obtained by adding and dissolving 0.04% by mass of sodium hydroxide in a proper amount of purified water was added to the mixture under stirring using a paddle mixer (manufactured by Nikko Chemicals Co., Ltd.). A purified water was added up to 100% by mass of the total amount of the preparation, and the mixture was stirred at room temperature until cool to 30° C. or lower.

The obtained preparation was in a form of a cream and it was confirmed with a polarizing microscope that the preparation had a lamellar liquid crystal structure.

### Example 5

0.1% by mass of adapalene, 0.1% by mass of methylparaben, 25% by mass of glycerin, 0.3% by mass of a carboxyvinyl polymer, 0.03% by mass of disodium edetate hydrate and a proper amount of purified water were mixed and heated at a temperature of 80° C. or higher (aqueous phase). Next, 5% by mass of a mixture of polyoxyethylene arachyl ether and stearyl alcohol (WAX230 available from Nikko Chemicals Co., Ltd.), 0.1% by mass of propylparaben, 1% by mass of glycerin monostearate and 5% by mass of squalane were mixed and dissolved by heating at a temperature of 80° C. or higher (oil phase). The oil phase was added to the aqueous phase in the state that is heated at 80-90° C. and stirred with a homogenizing mixer (manufactured by PRIMIX Corporation), followed by stirring for three minutes at 3500 rpm to emulsify. Then, a solution obtained by adding and dissolving 0.03% by mass of sodium hydroxide in a proper amount of purified water was added to the mixture under stirring using a paddle mixer (manufactured by Nikko Chemicals Co., Ltd.). A purified water was added up to 100% by mass of the total amount of the preparation, and the mixture was stirred at room temperature until cool to 30° C. or lower.

The obtained preparation was in a form of a cream and it was confirmed with a polarizing microscope that the preparation had a lamellar liquid crystal structure.

### Example 6

0.1% by mass of adapalene, 0.1% by mass of methylparaben, 25% by mass of glycerin, 0.4% by mass of a carboxyvinyl polymer, 0.03% by mass of disodium edetate hydrate and a proper amount of purified water were mixed, and heated at a temperature of 80° C. or higher (aqueous phase). Next, 5% by mass of a mixture of polyoxyethylene arachyl ether and stearyl alcohol (WAX230 available from Nikko Chemicals Co., Ltd.), 0.1% by mass of propylparaben and 5% by mass of squalane were mixed and dissolved by heating at a temperature of 80° C. or higher (oil phase). The oil phase was added to the aqueous phase in the state that is heated at 80-90° C. and stirred with a homogenizing mixer (manufactured by PRIMIX Corporation), followed by stirring for three minutes at 3500 rpm to emulsify. Then, a solution obtained by adding and dissolving 0.04% by mass of sodium hydroxide in a proper amount of purified water was added to the mixture under stirring using a paddle mixer (manufactured by Nikko Chemicals Co., Ltd.). A purified water was added up to 100% by mass of the total amount of the preparation, and the mixture was stirred at room temperature until cool to 30° C. or lower.

The obtained preparation was in a form of a cream and it was confirmed with a polarizing microscope that the preparation had a lamellar liquid crystal structure.

### Comparative Example 1

To a proper amount of purified water was added and dissolved 0.1% by mass of disodium edetate hydrate, and thereto was added 1.1% by mass of a carboxyvinyl polymer, followed by sufficient dispersing until a mass disappeared. Next, thereto was added a mixture obtained by adding 0.1% by mass of adapalene and 0.2% by mass of methylparaben to 4% by mass of propylene glycol and heating at a temperature of 80° C. or higher and mixed to be miscible. Then, thereto was added 0.2% by mass of polyoxyethylene (20) polyoxypropylene (20) glycol and mixed to be miscible, and further a solution obtained by adding and dissolving 0.18% by mass of sodium hydroxide in a proper amount of purified water was added to the mixture. A purified water was added up to 100% by mass of the total amount of the preparation and mixed until the mixture became uniform.

The obtained preparation was in a form of a white gel.

### Test Example 1

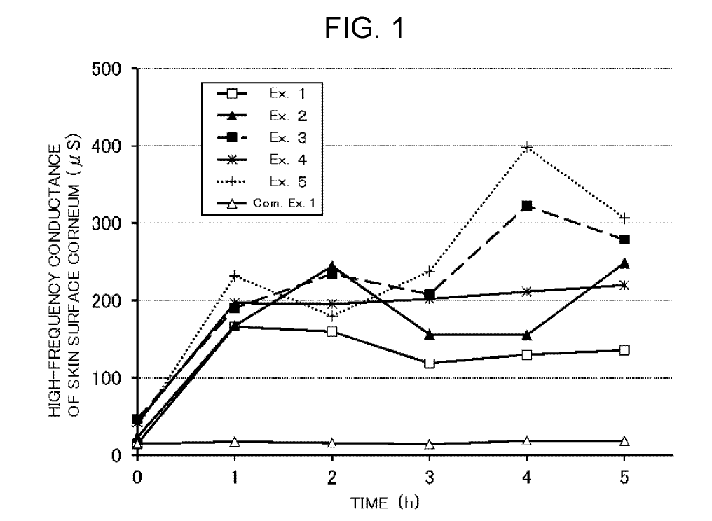

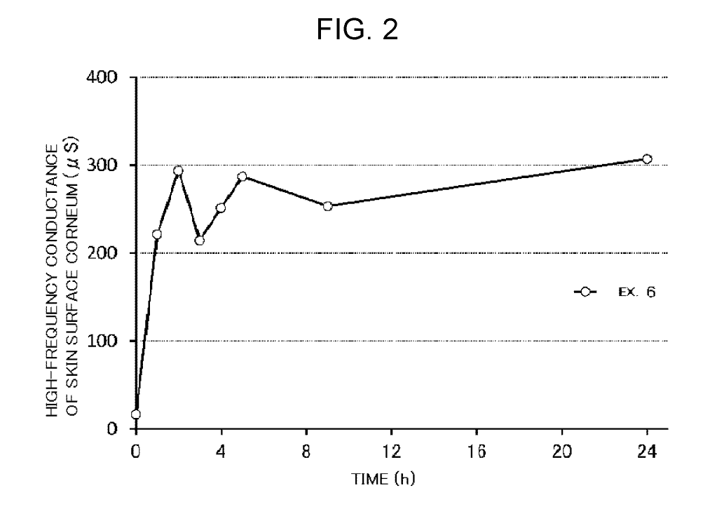

Each of the preparations for external use of Examples 1 to 6 and Comparative Example 1 was applied to a skin of the same person in an amount of 5 mg/3.14 cm² in an open system (n=3), and a high-frequency conductance of skin surface corneum at the applied portion was measured with a skin surface hygrometer SKICON-200 (manufactured by I.B.S. Co., Ltd.). The measurement of the high-frequency conductance of skin surface corneum was made during a time period of 1 to 24 hours after the application, assuming that a point of time just before the application is zero hour. The results are shown in FIG. 1 and FIG. 2.

Compounding formulations (% by mass) of Examples and Comparative Example 1 are shown in Table 1 for the purpose of comparison.

TABLE 1

| Compounded amount (% by mass) | Ex. 1 | Ex. 2 | Ex. 3 | Ex. 4 | Ex. 5 | Ex. 6 | Com. Ex. 1 |
|---|---|---|---|---|---|---|---|
| Adapalene | 0.1 | 0.1 | 0.1 | 0.1 | 0.1 | 0.1 | 0.1 |
| Propylene glycol | — | — | — | — | — | — | 4 |
| Carboxyvinyl polymer | 0.3 | 0.3 | 0.3 | 0.4 | 0.3 | 0.4 | 1.1 |
| Concentrated glycerin | 20 | 30 | 25 | 25 | 25 | 25 | — |
| Wax-230* | 5 | 5 | 8 | 5 | 5 | 5 | — |
| Glycerin monostearate | — | — | — | — | 1 | — | — |
| Polyoxyethylene (20) | — | — | — | — | — | — | 0.2 |
| polyoxypropylene (20) glycol | | | | | | | |
| Cholesterol | — | — | — | 0.1 | — | — | — |
| Methylparaben | 0.2 | 0.2 | 0.1 | 0.1 | 0.1 | 0.1 | 0.2 |
| Propylparaben | 0.1 | 0.1 | 0.1 | 0.1 | 0.1 | 0.1 | — |
| Squalane | 5 | 10 | 5 | 5 | 5 | 5 | — |
| Disodium edetate hydrate | 0.1 | 0.1 | 0.03 | 0.03 | 0.03 | 0.03 | 0.1 |
| Sodium hydroxide | 0.045 | 0.045 | 0.03 | 0.04 | 0.03 | 0.04 | 0.18 |
| Purified water | proper | proper | proper | proper | proper | proper | proper |
| | amount | amount | amount | amount | amount | amount | amount |

*A mixture of polyoxyethylene arachyl ether and stearyl alcohol available from Nikko Chemicals Co., Ltd.

From FIG. 1, it is seen that in Examples 1 to 5 according to the invention of the instant application, the conductance thereof was six or more times as high as that of the adapalene preparation of Comparative Example 1 even five hours after the application. When considering that generally a conductance before application of the preparations is about 20 μS, the result is surprisingly good, and it is seen that the compounding formulations of the preparations according to the invention of the instant application have a remarkably excellent moisture retention.

From FIG. 2, it is seen that in Example 6, high conductance is maintained even 24 hours after the application. It can be considered that one reason for exhibiting this effect of the invention of the instant application is such that the preparation of the invention of the instant application has a lamellar liquid crystal structure, and a liquid oily ingredient, a moisturizing ingredient and water can be held continuously on a skin, while the present invention is not intended to be bound by this theory.

Accordingly, the medical dermatological preparation for external use of the present invention can effectively reduce occurrence and degree of skin irritation attributable to an active drug.

### Example 7

0.1% by mass of adapalene, 0.1% by mass of methylparaben, 30% by mass of glycerin, 0.3% by mass of a carboxyvinyl polymer, 0.1% by mass of disodium edetate hydrate and a proper amount of purified water were mixed and heated at a temperature of 80° C. or higher (aqueous phase). Next, 5% by mass of a mixture of polyoxyethylene arachyl ether and stearyl alcohol (WAX230 available from Nikko Chemicals Co., Ltd.), 5% by mass of squalane and 0.05% by mass of propylparaben were mixed and dissolved by heating to a temperature of 80° C. or higher (oil phase). The oil phase was added to the aqueous phase in the state that is heated at 80-90° C. and stirred with a homogenizing mixer (manufactured by PRIMIX Corporation), followed by stirring for three minutes at 3500 rpm to emulsify. Then, a solution obtained by adding and dissolving 0.03% by mass of sodium hydroxide in a proper amount of purified water was added to the mixture under stirring using a paddle mixer (manufactured by Nikko Chemicals Co., Ltd.). A purified water was added up to 1.00% by mass of the total amount of the preparation, and the mixture was stirred at room temperature until cool to 30° C. or lower.

The obtained preparation was in a form of a cream and it was confirmed with a polarizing microscope that the preparation had a lamellar liquid crystal structure.

### Example 8

0.1% by mass of adapalene, 0.1% by mass of methylparaben, 30% by mass of glycerin, 0.3% by mass of a carboxyvinyl polymer, 0.1% by mass of disodium edetate hydrate and a proper amount of purified water were mixed and heated at a temperature of 80° C. or higher (aqueous phase). Next, 5% by mass of a mixture of polyoxyethylene arachyl ether and stearyl alcohol (WAX230 available from Nikko Chemicals Co., Ltd.), 5% by mass of squalane, 0.05% by mass of propylparaben and 1% by mass of batyl monostearate (GM-18SV manufactured by Nikko Chemicals Co., Ltd.) were mixed and dissolved by heating to a temperature of 80° C. or higher (oil phase). The oil phase was added to the aqueous phase in the state that is heated at 80-90° C. and stirred with a homogenizing mixer (manufactured by PRIMIX Corporation), followed by stirring for three minutes at 3500 rpm to emulsify. Then, a solution obtained by adding and dissolving 0.03% by mass of sodium hydroxide in a proper amount of purified water was added to the mixture under stirring using a paddle mixer (manufactured by Nikko Chemicals Co., Ltd.). A purified water was added up to 100% by mass of the total amount of the preparation, and the mixture was stirred at room temperature until cool to 30° C. or lower.

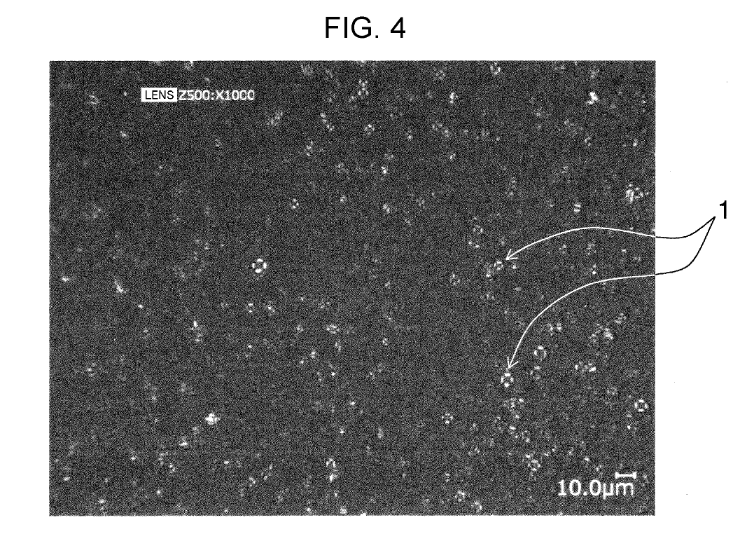

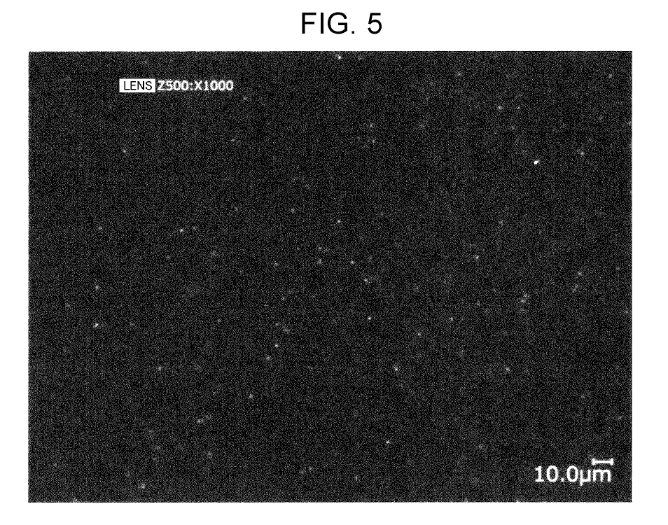

The obtained preparation was in a form of a cream and it was confirmed with a polarizing microscope that the preparation had a lamellar liquid crystal structure. FIG. 4 is a photographic image of the preparation observed and taken with a polarizing microscope (×1000) by placing 5 μL of the obtained preparation on a slide glass, covering the preparation with a cover glass and slowly pressing the cover glass from the top thereof for spreading the preparation to form it into a uniform thin film. The quadrangles with rounded corners are lamellar liquid crystals 1, and it is seen that there is a lot of lamellar liquid crystals as compared with a photographic image (FIG. 5) of a gel preparation of Comparative Example 1 taken similarly with a polarizing microscope.

### Example 9

0.1% by mass of adapalene, 0.1% by mass of methylparaben, 30% by mass of glycerin, 0.3% by mass of a carboxyvinyl polymer, 0.1% by mass of disodium edetate hydrate and a proper amount of purified water were mixed and heated at a temperature of 80° C. or higher (aqueous phase). Next, 5% by mass of a mixture of polyoxyethylene arachyl ether and stearyl alcohol (WAX230 available from Nikko Chemicals Co., Ltd.), 5% by mass of squalane, 0.05% by mass of propylparaben and 1% by mass of batyl isostearate (GM-18ISV manufactured by Nikko Chemicals Co., Ltd.) were mixed and dissolved by heating to a temperature of 80° C. or higher (oil phase). The oil phase was added to the aqueous phase in the state that is heated at 80-90° C. and stirred with a homogenizing mixer (manufactured by PRIMIX Corporation), followed by stirring for three minutes at 3500 rpm to emulsify. Then, a solution obtained by adding and dissolving 0.03% by mass of sodium hydroxide in a proper amount of purified water was added to the mixture under stirring using a paddle mixer (manufactured by Nikko Chemicals Co., Ltd.). A purified water was added up to 100% by mass of the total amount of the preparation, and the mixture was stirred at room temperature until cool to 30° C. or lower.

The obtained preparation was in a form of a cream and it was confirmed with a polarizing microscope that the preparation had a lamellar liquid crystal structure.

### Comparative Example 2

To 90% by mass of purified water was added 1.1% by mass of a carboxyvinyl polymer, followed by sufficient dispersing until a mass disappeared. Next, thereto was added a mixture obtained by adding 0.2% by mass of methylparaben to 2% by mass of propylene glycol and heating the mixture at a temperature of 80° C. or higher and mixed to be miscible. Thereafter, to the mixture was added a dispersion obtained by adding 0.2% by mass of polyoxyethylene (20) polyoxypropylene (20) glycol and 0.1% by mass of adapalene to 2% by mass of propylene glycol and fully dispersing until a mass disappeared. Further, thereto was added a solution obtained by adding 0.1% by mass of disodium edetate hydrate and 0.18% by mass of sodium hydroxide to 1.0% by mass of purified water. A purified water was added up to 100% by mass of the total amount of the preparation and mixed until the mixture became uniform.

The obtained preparation was in a form of a white gel.

### Test Example 2

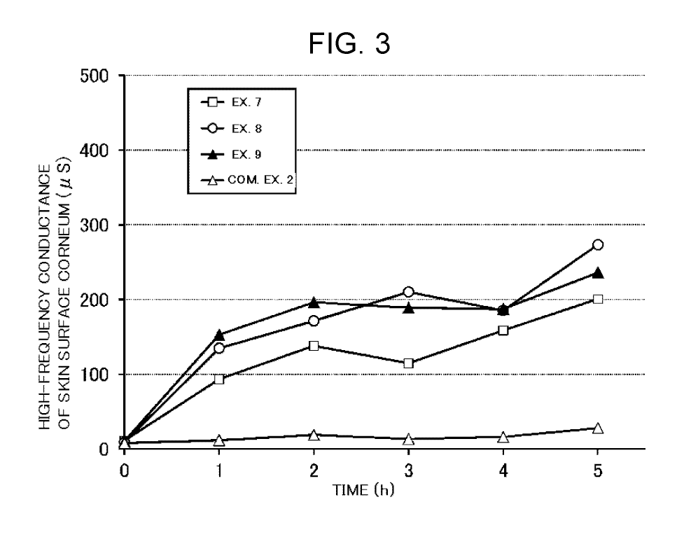

Each of the preparations for external use of Examples 7 to 9 and Comparative Example 2 was applied to a skin of the same person in an amount of 5 mg/3.14 cm² in an open system (n=3), and a high-frequency conductance of skin surface corneum at the applied portion was measured with a skin surface hygrometer SKICON-200 (manufactured by I.B.S. Co., Ltd.). The measurement of the high-frequency conductance of skin surface corneum was made every one hour during a time period of 1 to 5 hours after the application, assuming that a point of time just before the application is zero hour. The results are shown in FIG. 3.

Compounding formulations (% by mass) of Examples 7 to 9 and Comparative Example 2 are shown in Table 2 for the purpose of comparison.

TABLE 2

| | | | | Com. |
|---|---|---|---|---|
| Compounded amount (% by mass) | Ex. 7 | Ex. 8 | Ex. 9 | Ex. 2 |
| Adapalene | 0.1 | 0.1 | 0.1 | 0.1 |
| Propylene glycol | — | — | — | 4 |
| Carboxyvinyl polymer | 0.3 | 0.3 | 0.3 | 1.1 |
| Concentrated glycerin | 30 | 30 | 30 | — |
| WAX-230 * | 5 | 5 | 5 | — |
| Batyl monostearate | — | 1 | — | — |
| Batyl isostearate | — | — | 1 | — |
| Polyoxyethylene (20) | — | — | — | 0.2 |
| polyoxypropylene (20) glycol | | | | |
| Methylparaben | 0.1 | 0.1 | 0.1 | 0.2 |
| Propylparaben | 0.05 | 0.05 | 0.05 | — |
| Squalane | 5 | 5 | 5 | — |
| Disodium edetate hydrate | 0.1 | 0.1 | 0.1 | 0.1 |
| Sodium hydroxide | 0.03 | 0.03 | 0.03 | 0.18 |
| Purified water | proper | proper | proper | proper |
| | amount | amount | amount | amount |

* A mixture of polyoxyethylene arachyl ether and stearyl alcohol available from Nikko Chemicals Co., Ltd.

From FIG. 3, it is seen that in Examples 7 to 9 according to the invention of the instant application, the conductance thereof was ten or more times as high as that of the adapalene preparation of Comparative Example 2 even five hours after the application. When considering that the conductance before application of the preparation in Test Example 2 (application time: 0 hour) is about 10 S, the result is surprisingly good, and it is seen that the compounding formulations of the preparations according to the invention of the instant application has a remarkably excellent moisture retention similarly to Examples 1 to 6. Further, it is seen that in Examples 8 and 9, where the emulsion stabilizer was added, moisture retention as a whole is excellent as compared with Example 7, where an emulsion stabilizer was not added. It can be considered that one reason therefor is such that when the emulsion stabilizer is added, emulsified particles are not collapsed, are held on a skin for a long period of time and remain on the skin without evaporation of water droplets, thereby maintaining a moisture content on the skin. It is a matter of course that the present invention is not intended to be bound by this theory.

Accordingly, the medical dermatological preparation for external use of the present invention can effectively reduce occurrence and degree of skin irritation attributable to an active drug.

### EXPLANATION OF SYMBOL

1 Lamellar liquid crystal

1. A medical dermatological preparation for external use comprising an active drug, polyoxyethylene arachyl ether, stearyl alcohol, a liquid oily ingredient, a moisturizing ingredient and water.

2. The medical dermatological preparation for external use of claim 1, wherein the active drug is adapalene.

3. The medical dermatological preparation for external use of claim 1, wherein the liquid oily ingredient is at least one selected from the group consisting of squalane, liquid paraffin, jojoba oil, isopropyl myristate and isopropyl palmitate.

4. The medical dermatological preparation for external use of claim 1, wherein the moisturizing ingredient is at least one selected from the group consisting of glycerin, 1,3-butylene glycol, dipropylene glycol, polyethylene glycol, sorbitol, urea, glycolic acid, heparinoids, pyrrolidone carboxylic acid, collagen, γ-orizanol, γ-linolenic acid, linoleic acid, vitamin E, vitamin D, vitamin A, cholesterol, glucosamine, sodium hyaluronate, sodium chondroitin lactate, casein, glucose, fructose, trehalose, maltose, pullulan, erythritol, hydrolyzed fibroin, hydrolyzed collagen, maltitol and saccharose.

5. The medical dermatological preparation for external use of claim 1, further comprising an emulsion stabilizer.

6. The medical dermatological preparation for external use of claim 5, wherein the emulsion stabilizer is at least one selected from the group consisting of cetanol, cetostearyl alcohol, behenyl alcohol, batyl alcohol, batyl isostearate and batyl monostearate.

7. The medical dermatological preparation for external use of claim 1, which is in a dosage form of a cream, a gel or a lotion.
